(19) United States
(12) Patent Application Publication
(10) Pub. No.: US 2020/0221958 A1
(43) Pub. Date: Jul. 16, 2020

(54) SYSTEM FOR MEASURING HEART RATE

(71) Applicant: Sports Data Labs, Inc., Royal Oak, MI (US)

(72) Inventors: Viprali Bhatkar, Arlington, MA (US); Vivek Khare, Cupertino, CA (US); Stan Mimoto, Bethel Island, CA (US); Mark Gorski, Royal Oak, MI (US)

(73) Assignee: Sports Data Labs, Inc., Royal Oak, MI (US)

(21) Appl. No.: 16/246,923

(22) Filed: Jan. 14, 2019

(51) Int. Cl. A61B 5/0245; A61B 5/044; A61B 5/0456; A61B 5/00

(52) U.S. Cl. CPC A61B 5/0245; A61B 5/6801; A61B 5/0456; A61B 5/044

(57) ABSTRACT

A method of computing a hear rate compensates for noise in data transmission that can lead to false R-peak detection or missed R-peak detection. The method computes an updated heart rate based on a window of approximately ten seconds of digitized ECG readings broadcast from a sensor. A new value is calculated approximately every second such that the window of ECG readings overlaps considerably between consecutive calculations. The method compensates for noisy data by discarding heart rate samples that differ by more than a threshold amount from a previously calculated heart rate value. The threshold is adjusted based on a standard deviation of differences between heart rate samples.

### TECHNICAL FIELD

This disclosure relates to the field of human data monitoring systems. More particularly, the disclosure pertains to a system for measuring a heart rate, for example of a person engaged in sports or other highly active and mobile activity.

### BACKGROUND

Heart Rate (HR) is a key indicator of the function and performance of the heart during various activities. A real-time HR computation reflects beat by beat changes in HR due to underlying physical and/or mental activities. The changes in HR can be captured as Heart Rate Variability (HRV) and are very important in the diagnosis and monitoring of the heart health. Displaying an instantaneous HR during such activities provides important information about heart health as well as the effect of the underlying activity.

Heart rate computations can be adversely affected by the motion artifacts induced in the Electro-CardioGram (ECG) signal due to body movements. Some of these changes can be filtered out but when there are no R-peaks identified, sudden increase or decrease in the HR may be observed. Such frequent noisy periods in the raw signal can lead to a noisy HR waveform.

### SUMMARY OF THE DISCLOSURE

A system for measuring a heart rate includes a sensor, a server, and a display device. The sensor is configured to measure electric signals in the subject's body, convert analog measurements to digital readings, and transmit the digital readings. The server receives the digital readings and calculates heart rate based on overlapping segments of the digital readings by (i) identifying R-peaks within the segments, (ii) calculating a number of sample values based on times between adjacent R-peaks, (iii) discarding samples that are influenced by false peak detection or missed peak detection, and (iv) calculating an average of remaining sample values. The display device displays the averages of the remaining sample values. The server may determine that samples are influenced by false peak detection or missed peak detection in response to a sample value differing from a previous heart rate value by more than a first threshold. If a standard deviation of differences between samples is greater than a second threshold, the server may determine that samples are influenced by false peak detection or missed peak detection in response to the sample value differing from the previous heart rate value by more than a third threshold less than the first threshold.

A system for measuring a heart rate includes a sensor, a server, and a display device. The sensor is adapted for fixation to a subject's skin and configured to measure electric signals in the skin, convert analog measurements to digital readings, and transmit the digital readings. The server receive the digital readings and calculate heart rate values based on overlapping segments of the digital readings by (i) identifying R-peaks within the segments, (ii) calculating a number of sample values based on times between adjacent R-peaks, (iii) selecting samples within a first threshold of a previous heart rate value, and (iv) setting a current heart rate value to an average of the selected samples. Each sample value may be proportional to a reciprocal of a time between adjacent R-peaks. The server may select samples within a second threshold of the previous heart rate value in response to a standard deviation of differences between consecutive samples being greater than a third threshold. The server may set the current heart rate value equal to the previous heart rate value in response to the number of samples being less than a fourth threshold or in response to no samples being selected. The display device displays current heart rate values. The system may operate in near real time wherein the display device displays each current heart rate value before a respective succeeding heart rate value is calculated and the server calculates each current heart rate value before the sensor completes measuring all of the readings used to calculate the succeeding heart rate value. The server may compute an initial heart rate value by receiving a preliminary segment of the digital readings longer than the overlapping segments, identifying R-peaks within the preliminary segment, calculating sample values based on times between adjacent R-peaks, and calculating an average of the samples.

A method for measuring a heart rate of a person includes receiving readings from a sensor, processing the readings, and displaying the result. A first segment of readings is received from the sensor. R-peaks within the first segment are then identified. Then, a first plurality of sample values is calculated based on times between adjacent R-peaks. For example, a constant may be divided by times between adjacent R-peaks. A first subset of the first plurality of sample values are selected including only sample values within a first threshold of a previous heart rate value. Then, a first updated heart rate value is calculated based on an average of the first subset of sample values. The first updated heart rate value is then displayed. In later iterations, a second segment of the digital readings may be received from the sensor. A third segment of digital reading may be formed by appending the second segment to the first segment. R-peaks within the third segment may then be identified. A second plurality of sample values may be calculated based on times between adjacent R-peaks. Then, a plurality of differences between consecutive samples may be calculated. In response to a standard deviation of the differences exceeding a second threshold, a second subset of the second plurality of sample values may be selected including only sample values within a third threshold of the first updated heart rate value. A second updated heart rate value may then be calculated and displayed based on an average of the second subset of sample values. An initial heart rate value may be calculated based on a preliminary segment of the digital readings.

### BRIEF DESCRIPTION OF THE DRAWINGS

FIG. 1 is a schematic diagram of a heart rate measurement and display system.

FIG. 2 is a graph illustrating ECG measurements for a person with an increasing heart rate.

FIG. 3 is a flow chart for a method of calculating a heart rate value based on a stream of digitized ECG measurements in the system of FIG. 1.

FIG. 4 is a flow chart for performing the initialization step in the method of FIG. 3.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present invention. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

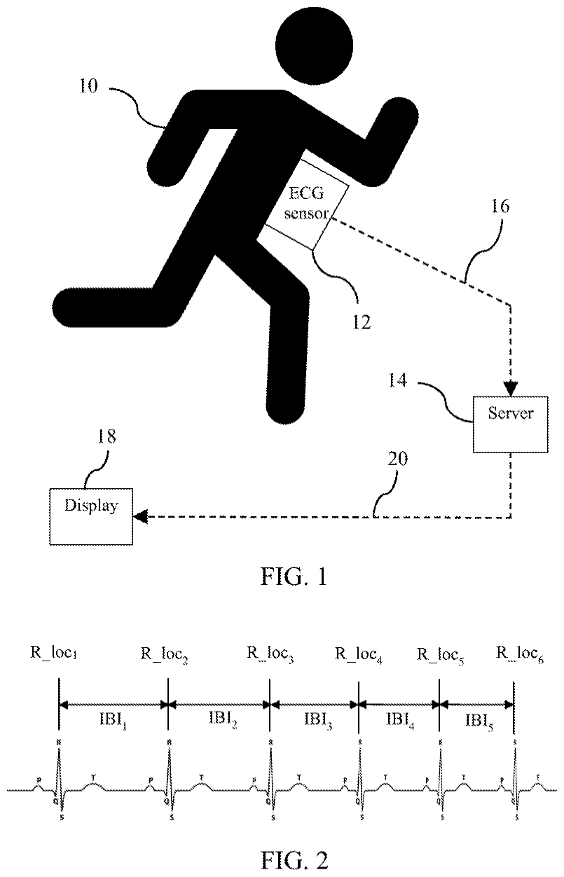

FIG. 1 illustrates a system for determining and displaying a heart rate of an active person 10, such as a person engaged in a sport. Depending on the nature of the activity, the person's heart rate may vary considerably over relatively short time frames. An ECG sensor 12 is attached to, or embedded within, the person 10 and measures electrical changes in the person's skin associated with heart function. The sensor digitizes these measurements and transmits the digitized measurements to a server 14 using a wireless communication protocol 16 such as Bluetooth or Will. The communications protocol may be direct or may involve intermediate devices as long as the measurements reach the server in near real-time. Preferably, between 250 and 1000 such measurements are broadcast per second. From these measurements, the server 14 computes a heart rate value approximately once per second. The server 14 communicates the heart rate values to a display 18 using a protocol 20. The display may be arranged to be viewed by the person 10 and/or by others. Protocol 20 may be a wireless protocol or a wired protocol. In some embodiments, the display 18 and the server 14 may be integrated into a single physical device such as a smartphone with processing and display capabilities. The server 14 and/or the display 18 may be wearable by the person 10, mobile but not worn by the person, or stationary.

FIG. 2 illustrates an exemplary output of the ECG measurements. Note that the measurements follow a regular pattern that repeats for each heart beat. Various points in the repeating pattern are labelled P, Q, R, S, and T. The R point is indicated by a localized peak. The time of the R peaks are labelled R_locᵢ for 1≤i≤n. The difference between successive R_loc times are labelled Inter-Beat Interval i (IBIᵢ) for 1≤i<n. (For the output illustrated in FIG. 2, n=6.) Note that the time between R peaks is shorter near the end of the time period illustrated in FIG. 2 than near the beginning of the interval. This indicates that the individual's heart rate is increasing. Calculating the heart rate when the heart rate is rapidly changing is more difficult than calculating it when the heart rate is steady. Although the graph of FIG. 2 shows very distinct R peaks, actual measurements are not necessarily so clear. In the system of FIG. 1, there are a variety of sources of noise in the ECG signal, such as measurement noise from movement of the person's body and transmission noise.

One of the ways abnormalities in an instantaneous HR can be handled is by a windowing method. A detailed description of the method is described in the sections below. A sliding window of a certain duration such as 10-sec to 5-minutes can be used to look at the ECG data. It contains several detected R-peaks (beats). In a noisy signal, some of these beats could be noise peaks resulting in a very high or low HR outliers. By analyzing the distribution of these values, it is possible to accept or reject a good beat before the HR computations. For the finer resolution and to use the past HR information, overlapping sliding windows can be used. This method is very useful and effective in avoiding abrupt changes in HR.

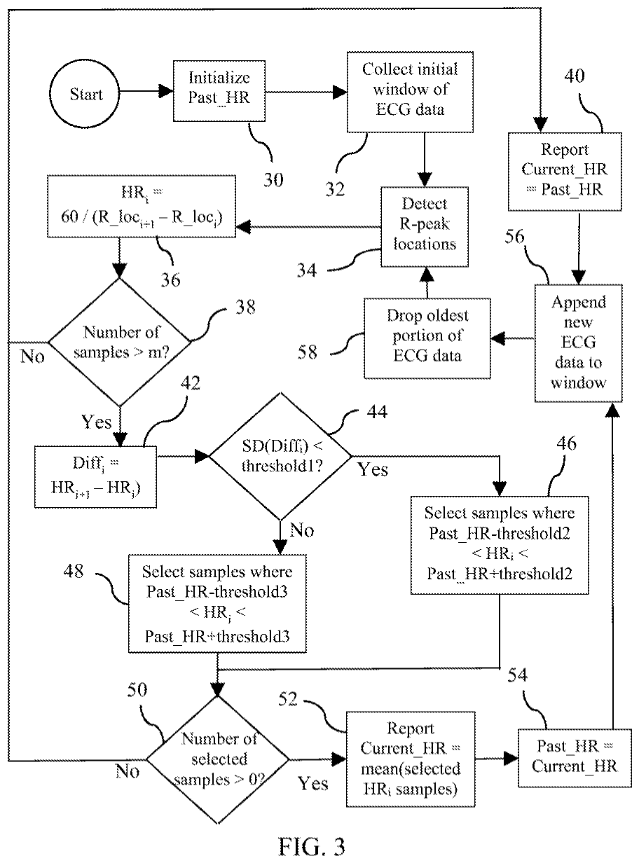

FIG. 3 illustrates a method, executed by server 14, of computing a stream of heart rate values based on the received stream of digitized ECG measurements. The method utilizes a parameter, Past_HR, representing the most recently calculated heart rate value. At 30, this parameter is initialized. The procedure for initializing Past_HR is described in more detail below. At 32, the method collects approximately ten seconds of ECG data. The precise duration of data to collect is a tunable parameter. Using a longer period will make the method less responsive to rapid changes in heart rate. Using a period that is too short may increase the frequency of the method not computing an updated heart rate. At 34, the R-peak locations are identified. Various methods are known for this step, including the Pan-Thompkins algorithm which is recommended by the inventors. The result of this step is a series of times, R_locᵢ, 1≤i≤n. At 36, the method calculates a number of sample values based on the time between adjacent R_loc values. Specifically, each sample value, HRᵢ for 1≤i≤n−1, is equal to 60 divided by the time difference between adjacent R_loc values. The HRᵢ sample values have the same units, beats per minute, as the person's heart rate and would be expected to fall in the same general range. Some embodiments may use different but related sample values, such as IBI, with appropriate conversions before reporting. At 38, the method tests whether the number of samples exceeds a predefined minimum, such as 10. The required number of samples is a tunable parameter. If an insufficient number of samples is available, then the method branches to 40 and reports the previous value without computing an updated value.

Two types of peak detection errors may occur due to noise in the received time series of digitized ECG measurements. The first type of error is when a true peak is not detected. The consequence of this type of error is an IBI value equal to the sum of two correct IBI values. The resulting HR; value will be substantially smaller than the correct heart rate. The second type of error is when a false peak is detected. The consequence of this type of error is two IBI values that add up to a correct IBI value. The resulting two HRᵢ values are each larger than a correct heart rate. For either type of error, the resulting false values should not be included in the calculation of a reported heart rate value.

If the number of samples is sufficient, the method selects a subset of the samples that are within a threshold of Past HR. The threshold is dependent upon the standard deviation of the differences of the samples. At 42, the differences between adjacent samples, Diffᵢ for 1≤i<n−2, are calculated. At 44, the standard deviation of the differences in HRᵢ samples is computed and compared to a first threshold. The inventors recommend a value of 5 beats per minute for this first threshold, although this is a tunable parameter. If the standard deviation is less than the first threshold, then samples are selected at 46 based on whether or not they are within a second threshold of Past_HR. The inventors recommend a value of 20 beats per minute for this second threshold, although this is a tunable parameter. If the standard deviation is greater than or equal to the first threshold at 44, then samples are selected at 48 based on whether or not they are within a third threshold of Past_HR. The inventors recommend a value of 12 beats per minute for this third threshold, although this is a tunable parameter. At 50, the method tests whether any samples have been selected. If not, then the method branches to 40 and reports the previous value without computing an updated value.

If some samples are selected at 50, then the method computes the Current_HR at 52 by taking an average, preferably the mean, of the selected samples. This updated heart rate value is then reported by transmitting it to the display unit. At 54, Past_HR is set equal to Current_HR. The updated value will be the basis for selecting samples in future iterations.

After reporting either an updated value at 54 or reporting a previous value at 40, the method collects approximately one second of additional ECG data at 56 and appends the additional data to the end of the current ECG data window, which may be called a segment. At 58, the oldest portion of ECG data, equal in duration to the data added at 56, is dropped from the ECG data window. The time interval at 56 and 58 may be adjusted if values are desired more or less frequently. As a result, approximately 90% of the data from the previous iteration is included in the new ECG data window.

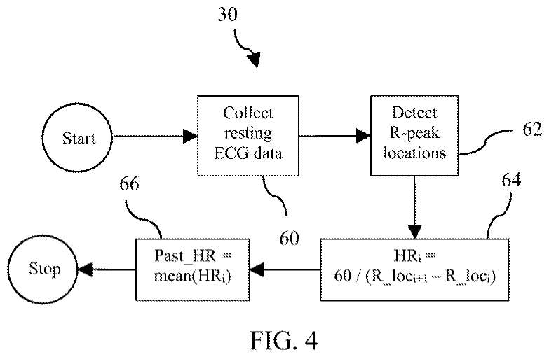

FIG. 4 describes the initialization procedure of step 30. At 60, approximately two minutes of ECG data is collected by the server. Preferably, this data is collected while the person is at rest so that the heart rate is relatively constant and the person's motion does not lead to increased transmission noise. At 62, the method identifies the R peaks in the collected stream of ECG measurements using, for example, the Pan-Thompkins algorithm. At 64, a number of sample HRᵢ values are computed from the times of the R peaks, R_locᵢ. At 66 the initial Past_HR value is computed by taking an average of the sample of HRᵢ values.

Over past twenty years, heart rate has been widely used in medical as well as consumer health monitoring systems. Heart rate and monitoring is effectively used in sports for the training and for the evaluation of the performance. Heart rate is a non-invasive measure of autonomic nervous system (ANS). Heart rate is also used in a wide variety of applications such as optimizing training and recovery, identify risk of disease, mortality and morbidity, etc.

Near real-time remote reporting of heart rate may be used in a number of applications. For example, a trainer may adjust an athlete's exercise pattern during training based on the computed heart rate. During training or competition, an athlete may be rested based on a heart rate indicating sub-optimal performance or risk of injury. The heart rate data may be the subject of wagers in sports betting applications.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. As such, embodiments described as less desirable than other embodiments or prior art implementations with respect to one or more characteristics are not outside the scope of the disclosure and can be desirable for particular applications.

What is claimed is:

1. A system for measuring a heart rate, the system comprising:
a sensor configured to measure electric signals in a subject's body, convert analog measurements to digital readings, and transmit the digital readings;
a server configured to receive the digital readings and calculate heart rate based on overlapping segments of the digital readings by identifying R-peaks within the segments, calculating a number of sample values based on times between adjacent R-peaks, discarding samples that are influenced by false peak detection or missed peak detection, and calculating an average of remaining sample values; and
a display device configured to display the averages of the remaining sample values.

2. The system of claim 1 wherein the server determines that samples are influenced by false peak detection or missed peak detection in response to a sample value differing from a previous heart rate value by more than a first threshold.

3. The system of claim 2 wherein, in response to a standard deviation of differences between samples being greater than a second threshold, the server determines that samples are influenced by false peak detection or missed peak detection in response to the sample value differing from the previous heart rate value by more than a third threshold different than the first threshold.

4. The system of claim 3 wherein the third threshold is less than the second threshold.

5. A system for measuring a heart rate, the system comprising:
a sensor adapted for fixation to a subject's skin and configured to measure electric signals in the skin, convert analog measurements to digital readings, and transmit the digital readings;
a server configured to receive the digital readings and calculate heart rate values based on overlapping segments of the digital readings by identifying R-peaks within the segments, calculating a number of sample values based on times between adjacent R-peaks, selecting samples within a first threshold of a previous heart rate value, and setting a current heart rate value to an average of the selected samples; and
a display device configured to display current heart rate values.

6. The system of claim 5 wherein the display device displays each current heart rate value before a respective succeeding heart rate value is calculated.

7. The system of claim 6 wherein the server calculates each current heart rate value before the sensor completes measuring all of the readings used to calculate the succeeding heart rate value.

8. The system of claim 5 wherein the server selects samples within a second threshold of the previous heart rate value in response to a standard deviation of differences between consecutive samples being greater than a third threshold.

9. The system of claim 5 wherein the server sets the current heart rate value equal to the previous heart rate value in response to the number of samples being less than a fourth threshold.

10. The system of claim 5 wherein the server sets the current heart rate value equal to the previous heart rate value in response to no samples being selected.

11. The system of claim 5 wherein each sample value is proportional to a reciprocal of a time between adjacent R-peaks.

12. The system of claim 5 wherein the server computes an initial heart rate value by receiving a preliminary segment of the digital readings longer than the overlapping segments, identifying R-peaks within the preliminary segment, calculating sample values based on times between adjacent R-peaks, and calculating an average of the samples.

13. A method for measuring a heart rate of a person, the method comprising:
receiving a first segment of digital readings from a sensor;
identifying R-peaks within the first segment;
calculating a first plurality of sample values based on times between adjacent R-peaks;
selecting a first subset of the first plurality of sample values including only sample values within a first threshold of a previous heart rate value;
calculating a first updated heart rate value based on an average of the first subset of sample values; and
displaying the first updated heart rate value.

14. The method of claim 13 further comprising:
receiving a second segment of the digital readings from the sensor;
forming a third segment of digital reading by appending the second segment to the first segment;
identifying R-peaks within the third segment;
calculating a second plurality of sample values based on times between adjacent R-peaks;
selecting a second subset of the second plurality of sample values including only sample values within the first threshold of the first updated heart rate value;
calculating a second updated heart rate value based on an average of the second subset of sample values; and
displaying the second updated heart rate value.

15. The method of claim 13 further comprising:
receiving a second segment of the digital readings from the sensor;
forming a third segment of digital reading by appending the second segment to the first segment;
identifying R-peaks within the third segment;
calculating a second plurality of sample values based on times between adjacent R-peaks;
calculating a plurality of differences between consecutive samples;
in response to a standard deviation of the differences exceeding a second threshold, selecting a second subset of the second plurality of sample values including only sample values within a third threshold of the first updated heart rate value;
calculating a second updated heart rate value based on an average of the second subset of sample values; and
displaying the second updated heart rate value.

16. The method of claim 13 wherein calculating the first plurality of sample values comprises dividing a constant by times between adjacent R-peaks.

17. The method of claim 13 further comprising computing an initial heart rate value by:
receiving a preliminary segment of the digital readings;
identifying R-peaks within the preliminary segment;
calculating sample values based on times between adjacent R-peaks; and
calculating an average of the samples.
